(19) United States
(12) Patent Application Publication
(10) Pub. No.: US 2018/0338359 A1
(43) Pub. Date: Nov. 22, 2018

(54) SYSTEMS AND METHODS OF DYNAMIC ILLUMINATION AND TEMPORALLY COORDINATED SPECTRAL CONTROL AND BIOLOGICAL DIMMING

(71) Applicant: Biological Innovation & Optimization Systems, LLC, Melbourne, FL (US)

(72) Inventor: Robert Soler, San Marcos, CA (US)

(73) Assignee: Biological Innovation & Optimization Systems, LLC, Cambridge, MA (US)

(21) Appl. No.: 15/875,143

(22) Filed: Jan. 19, 2018

Related U.S. Application Data
(60) Provisional application No. 62/450,893, filed on Jan. 26, 2017.
(60) Provisional application No. 62/450,887, filed on Jan. 26, 2017.
(60) Provisional application No. 62/583,393, filed on Nov. 8, 2017.
(60) Provisional application No. 62/543,227, filed on Aug. 9, 2017.

(51) Int. Cl. H05B 33/08; H05B 37/02; A61N 5/06

(52) U.S. Cl. CPC H05B 33/0857; H05B 37/0281; A61N 5/0618; A61N 2005/0626; A61N 2005/0663; A61N 2005/0652; H05B 37/0272

(57) ABSTRACT

Lighting systems and methods for providing biologically optimized illumination throughout the day are disclosed. Systems and methods of providing LED light engines and associated illumination spectrums that are both visually appealing, rich in melanopic flux and that reduce blue light hazard exposure are disclosed. Embodiments of the invention relating to specific spectra of illumination containing high or low amounts of melanopic light, spectrally and spatially tunable LED lighting systems, programmed and automated controllers for temporally controlling bio-effective illumination, and dimming circuitry for tuning the spectral output of lighting devices are also disclosed.

### RELATED APPLICATIONS

This application claims priority to and the benefit of U.S. Provisional Application No. 62/450,887, filed Jan. 26, 2017, U.S. Provisional Application No. 62/450,893, filed Jan. 26, 2017, U.S. Provisional Application No. 62/543,227, filed Aug. 9, 2017, and U.S. Provisional Application No. 62/583,393, filed Nov. 8, 2017. The contents of each of the aforementioned patent applications are incorporated herein in their entireties. This application is also related to application Ser. No. 15/609,294, filed May 31, 2017, and application Ser. No. 15/833,023, filed Dec. 6, 2017, both of which are incorporated herein by reference in their entireties.

Except to the extent that any of the disclosure in the referenced patents conflicts with the disclosure herein, the following US patents and publications, which include inter alia disclosure pertaining to light emitting diodes, LED luminaires and light engines, color mixing, and LED driving and switching methods and systems, and their application in LED technologies and biological lighting are incorporated herein by reference in their entireties: U.S. Pat. Nos. 9,788,387, 9,844,116, 8,366,755 7,744,243, 7,317,403, 7,358,954, 8,736,036, 8,597,963, 6,635,987, and 20140232288.

### FIELD OF THE INVENTION

Embodiments of the invention relate generally to lighting systems and, methods for providing biologically optimized illumination throughout the day in which the illumination is varied, both spectrally and spatially in coordination with an individual's or population's circadian cycles in order to facilitate circadian rhythm regulation. Embodiments of the invention relate to specific spectra of illumination containing high or low amounts of melanopic light, spectrally and spatially tunable LED lighting systems, programmed and automated controllers for temporally controlling the bio-effective illumination, and dimming circuitry for tuning the spectral output of lighting devices.

### BACKGROUND OF THE INVENTION

Light emitting diode (LED) technology is a maturing technology that continues to show improvements in efficiency, customability and cost reduction. LED technology is rapidly being deployed in a host of industries and markets including general lighting for homes, offices, and transportation, solid state display lighting such as in LCDs, aviation, agricultural, medical, and other fields of application. The increased energy efficiency of LED technology compared with other lighting solutions coupled with the reduction of costs of LED themselves are increasing the number of LED applications and rate of adoptions across industries. While LED technology promises greater reliability, longer lifetimes and greater efficiencies than other lighting technologies, the ability to mix and independently drive different color LEDs to produce customized and dynamic light output makes LED technology and solid state lighting (SSL) in general robust platforms to meet the demands of a variety of market needs and opens the door to many new applications of these lighting technologies. The ability to tailor and tune the output spectra of LED fixtures and dynamically switch individual LEDs “on-the-fly”, for example in response to an environmental cue, dramatically opens up the application space of solid state lighting.

As is well known in the art, LED luminaires generally comprise one or more individual LEDs dies or packages mounted on a circuit board. The LEDs may be electrically connected together on a single channel or be distributed and electrically driven across multiple independent channels. The LEDs are typically powered by current from an associated LED driver or power supply. Examples of these power supply drivers include AC/DC and DC/DC switched mode power supplies (SMPS). Examples of LED power drivers include power supplies designed to supply constant current to the LED string in order to maintain a consistent and steady light output from the LEDs. LEDs may also be powered by an AC power source. Direct AC power typically undergoes rectification and other power conditioning prior to being deliver to the LEDs. LED luminaires may also comprise an optic or diffuser, a heat sink and other structural components.

Although LEDs may be combined in such a way to deliver a wide variety of specific color outputs, LED luminaires for general lighting typically are designed to produce white light. Light perceived as white or near-white may be generated by a combination of red, green, and blue (RGB) LEDs. Output color of such a device may be altered by color mixing, for instance varying the amount of illumination produced by each of the respective color LEDs by adjusting the supply of current to each of the red, green, and blue LEDs. Another method for generating white or near-white light is by using a lumiphor such as a phosphor in conjunction with a blue “pump” LED. Still another approach for producing white light is to stimulate phosphors or dyes of multiple colors with an LED source. Many other approaches can also be taken.

FIG. 1a shows example spectral power distributions (SPDs) from conventional white light LEDs of three different correlated color temperatures. For each of these white light LED sources, the peak at around 450 nm represents the light contribution from a blue “pump” LED and the broader peak, for example and light above 500 nm, is due to the luminescence of one or more phosphors that have been excited by the blue light. In these conventional LED white light sources there is a trough of spectral power in the region around 490 nm.

LEDs, as with all manufactured products, have material and process variations that yield products with corresponding variation in performance. At present, LED manufacturers are challenged to produce uniform color points in their white LEDs and are limited to a “bandwidth spread” in their monochromatic LEDs as well. There are a number of reasons for this inability to achieve mass production of LEDs with uniform color points, key among them are related to the packaging of the LEDs. There may be considerable variability from LED to LED, particularly in the case of phosphor converted LEDs, since both the variability of the LED chip and the phosphor coating can introduce variability into the performance of the final packaged LED. While the manufacturers of the packaged LEDs typically “bin” the final packaged LEDs to provide products of similar light and color output, even LEDs in the same bin will exhibit variations in color output.

Although embodiments of the invention are not dependent on such, it is believed that the gap in spectral power output between 480 and 500 nm, with a trough around 490 nm, that exists in conventional white light LEDs (e.g., as shown in FIG. 2) is a result of the LED industry recognizing the challenges posed in color uniformity when employing light in the aforementioned region. The retinal response over this region (e.g., 480-500 nm), is such that the eye and visual system is extremely discriminative of light and light color in this spectral region. Additionally, LED manufacturers who make monochromatic LEDs, with a Full Width Half Maximum (FWHM) less than 40 nm, can typically only guarantee that any LED of a specific bin (i.e., within a certain color spectral bandwidth) will vary by no more than 5 nm in color output from another LED of the same bin. A lighting designer or manufacture attempting to construct a luminaire with a specific color output spectrum is challenged to provide a luminaire with consistent color output while using LEDs which may have an unacceptable wide range (e.g., 5 nm) of light output. Hence, because of the enhanced visual discrimination in the 480-500 nm color region, employing monochromatic LEDs in this region may result in unacceptable perceived color differences between LED fixtures that are designed to yield the same color output. Generating an LED spectrum with a consistent (x, y) color point while using monochromatic enhancement in the region from 480 nm-500 nm is a problematic challenge.

Melanopsin is a type of photopigment belonging to a larger family of light-sensitive retinal proteins called opsins, and is found in intrinsically photosensitive retinal ganglion cells (ipRGCs) of humans and other mammals. Melanopsin plays an important non-image-forming role in the photoentrainment of circadian rhythms as well as potentially many other physiologic functions. Stimulation of melanopsin-containing ipRGCs contributes to various reflexive responses of the brain and body to the presence of light. FIG. 1b shows the action spectrum of melanopsin 30 together with SPDs of conventional LED lights of different color temperatures 32. Melanopsin photoreceptors are sensitive to a range of wavelengths and reach peak light absorption at wavelengths around 480-500 (or 490) nanometers (nm).

Melanopic light, that is light corresponding to the melanopsin action spectrum, including particularly the wavelengths in the 480-500 nm region is important for non-visual stimuli including physiological and neurological effects such as pupillary light reflex and circadian entrainment and/or disruption. Time coordinated exposure, including over-exposure and under-exposure to melanopic light can be used to entrain and facilitate healthy circadian rhythms in humans and other mammals. When used herein, melanopic light is meant to generally refer to light that stimulates melanopsin and or that may have an effect on human circadian rhythms. When used herein, unless otherwise specified, “melanopic light” is not restricted to a particular or narrow band of wavelengths but rather is meant to mean light that corresponds to or is contained within range of wavelengths that correspond to the that melanopsin action spectrum. As shown in FIG. 1a conventional LED lighting fixtures provide less than optimal and potentially insufficient light in these biologically important wavelength ranges (e.g., non-visual stimulus) at standard light levels.

Blue Light Hazard”, as defined by ANSI/IESNA RP-27.3-07, is the potential for a photochemically induced retinal injury resulting from radiation exposure primarily between 400 nm and 500 nm. Scientific data indicates that blue light can cause excessive amounts of reactive oxygen species in the retina, which may result in cumulative oxidative stress which can cause inter alia accelerated cellular aging in the retina. FIG. 1b also illustrates the spectral region 34 associated with the blue light hazard. Even with conventional light levels, blue light exposure may cause long term damage over the course of years of exposure. This oxidative stress may be compounded and/or accelerated if the lighting illumination spectrum is deficient or depleted of light associated with non-visual stimulus. For example, the pupillary light reflex (PLR) is a reflex that controls the diameter of the pupil in response to the intensity (luminance) of light that falls on the retinal ganglion cells of the eye. This reflex thereby assists in, inter alia, adaptation to various levels of lightness or darkness. Insufficient stimulus of the RGCs, which may occur in the absence of sufficient melanopic light, that is light that falls within the melanopsin action spectrum region as shown in FIG. 1b and which provides the necessary stimulus of the RGCs, may result in reduced pupillary constriction, thereby allowing more blue light to enter the eye potentially resulting in increased and accelerated oxidative stress on the retina. As shown in FIG. 1b, conventional LED lighting fixtures provide peak emissions that overlap substantially with the blue light hazard spectral region.

As discussed above, light and in particular blue or bluish light may have both positive and negative effects on human circadian rhythms and regulation thereof depending on what type of light and how much light is received by the human visual system and the timing of such light exposure. Some lighting approaches use higher color temperatures as ways to maximize circadian impact. Examples of such color temperatures include 6500K, which correspond to daylight conditions. However, these 6500K spectrum LEDs are typically depleted of spectral energy in the 490 nm region and produce a large or heightened amount of 450 nm light. This conventional situation may pose health hazards including potential retinal damage because the conventional white light producing LEDs, which do not have continuity between the melanopic region and the blue light hazard region, may result in inappropriate pupillary dilation during exposure to potentially harmful blue light blue light.

The spatial distribution of illumination is also important with respect to human biological stimulation. Circadian related photoreceptors are in macular and peripheral vision nearest to the fovea. Melanopsin related photoreceptors are most sensitive in the lower hemisphere of the retina. Selective stimulation of these photoreceptors is possible by directing illumination, and specifically melanopic light, towards or away from the region of the retina where melanopic photoreceptors are most concentrated or most sensitive or responsive. If the desire is to optimally stimulate these photoreceptors, then a light source that produces high biological light (i.e., melanopic light) in this region would be a good solution. Equivalent Melanopic Lux (EML) is a metric for measuring the biological effects of light on humans. EML as a metric is weighted to the ipRGCs response to light and translates how much the spectrum of a light source stimulates ipRGCs and affects the circadian system. Melanopic ratio is the ratio of melanopic lux to photopic lux for a given light source.

Illumination emanating (e.g., reflecting) from vertical surfaces (e.g., upper portions of walls and ceilings) has a higher biological significance compared to lower horizontal surfaces (e.g., desktops and tabletops). This differential in biological effect is due at least in part to the fact that there is a greater concentration of melanopsin receptors (ipRGCs) in the lower hemisphere of the human retina than in the upper hemisphere. Specifics biological effects of light impacting the lower hemisphere of the retina may be greater than the biological effect of the same light incident on the upper hemisphere. Thus, optimizing biological effects of lighting requires the proper modulation of light and light distributions, not only in the spectral domain, but in the spatial domain as well.

While it is well known that the exposure to light, both natural and artificial, can affect an individual's circadian rhythms, it appears that the natural light of the sky during twilight, that is the short period around dawn or dusk when the sun is near the horizon, may have a significant impact on circadian drive and/or the gating of sleep pressure. Although the sky appears deep blue during twilight, it has significantly less radiant energy in the melanopic region (e.g., 490 nm) and significantly higher radiant energy in the 420 nm region, as compared to the sky during midday.

Although not well understood, recent scientific data indicates that the suprachiasmatic nucleus contains color representation of the sensed color of light. During the vast majority of the daytime, when the sun is up, the color temperature of the sky is between 5500 K and 7000K. The only time when this changes is during twilight periods when the sun is low. Common perception suggests that at these times the sky gets redder. However this is not the case, and while the sun appears redder as its irradiance travels through more of our earth's atmosphere, in fact the sky gets much bluer (e.g., at twilight, the color temperature of the sky may be at 8000-9000 K).

There are two unique and compelling circadian phenomenon, which coincide with the time when the sky gets bluer. First, sleep inertia, which is tendency for humans to remain asleep, occurs during sleep. Upon wakening, a circadian driven surge in blood cortisol levels helps individuals to wake up refreshed by mitigating sleep inertia. This cortisol response has been shown to synergistically occur with presence of light. On the other end of the day, e.g., at sunset, the wake maintenance zone portion of the circadian cycle has been demonstrated as a point of hyperactivity and enhanced neurobiological activity. It is hypothesized that this heightened activity may be an evolutionary survival response to insure individuals have sufficient alertness and energy to complete any tasks and find safety prior to the onset of darkness. At the time of day around twilight (or equivalent point in a circadian photoperiod) the human neurophysiology may be affected by specific light cues (that occur only at twilight) with regard to the body's circadian rhythm. For example, one effect may be the initiation of a sleep gating process (or conversely the absence or reduction of such gating without exposure to the twilight).

There is a need for general lighting device that delivers white light with excellent color rendering and esthetic characteristics and provides sufficient flux of melanopic light, generating sufficient spectral power in the relevant wavelengths to provide adequate non-visual stimulus associated with important physiological responses and functions. There is a need for lighting systems and methods that target and optimize biological effects by providing the appropriate lighting in both spatial and spectral domains. There is also a need to for lighting solutions that provide illumination that may be both spectrally and spatially modulated in order to target or optimize certain light sensitive biological effects. There is a need for lighting systems that create layers of light that illuminate different surfaces at different times of day (for example, high vertical illumination during biological daytime, and low vertical illumination during biological night time).

There is a need for lighting devices and systems which can provide appropriate biological lighting to individuals and groups of individual throughout the day or other photoperiods (e.g., circadian cycles), including lighting systems that can provide illumination with both increased amounts of melanopic light, for example during the daytime, and decreased or low amounts of melanopic light during other portions of the circadian photoperiod, for example at nighttime, in order to facilitate circadian rhythm regulation, improve sleep hygiene and contribute to the overall health. There is also a need for a lighting system that can simulate the lighting exposure of natural twilight which can stimulate one or more circadian gating mechanisms that coordinate with circadian drive and sleep pressures to maintain proper rhythmicity. There is a further need for a lighting device that provides high efficacy white light depleted of melanopic light for use at nighttime and/or as a nightlight that does not adversely impact circadian rhythms.

### BRIEF SUMMARY

Embodiments of the invention include an LED light engine for producing illumination with adequate amounts of melanopic light and for facilitating circadian rhythm regulation comprising a first LED module operable to produce white light illumination, and a second LED module operable to produce illumination with a first peak intensity between 470 nm and 500 nm and a second peak intensity between 640 nm and 680 nm wherein the second peak intensity is less than said first peak intensity. Embodiments also include an LED light engine comprising a third LED module operable to produce illumination with a first peak intensity between 470 nm and 500 nm and a second peak intensity between 410 nm and 430 nm and comprising electrical circuit means for connecting said first and second and third LED modules to a source of electrical power whereby the magnitude of electrical power supplied to said second and third LED modules may be varied thereby varying the intensity of the illumination output of said second and third LED modules. In some embodiments, the correlated color temperature of the output from the light engine when both the second LED module and third LED module are energized to illumination exceeds 7500 K and the first LED package produces white light with a correlated color temperature between 2500 K and 3500 K. In some embodiments, the full width of the peak at half its maximum intensity of said first peak intensity of the second LED package is less than 30 nm. In other embodiments, the light engine further comprises a nighttime LED module operable to emit light wherein the total radiant power emitted in a first wavelength band from 400 nm to 450 nm is greater than 10% of the total radiant power emitted and wherein the total radiant power emitted in a second wavelength band from 450 nm to 500 nm is less than 3% of the total radiant power emitted.

Other embodiments include a method of adjusting the spectral output of an LED light engine to facilitate circadian rhythm regulation comprising the steps of: providing a light engine comprising a first LED module and a second LED module wherein the first LED module produces white light and the second LED module produces light that has a maximum peak emission intensity between 470 nm and 490 nm and wherein the light engine contains means for adjusting electrical current supplied to said second LED module, and adjusting the current flow to said second LED package such that the intensity of light emitted from the light engine between 470 nm to 490 nm is increased during a first portion of a photoperiod and decreased during a second portion of the photoperiod. Embodiments include methods wherein said first portion of the photoperiod corresponds to circadian daytime and the current flow to the second LED package is adjusted to be at or near maximum thereby providing illumination rich in melanopic light and wherein said second portion of the photoperiod corresponds to circadian nighttime and the current flow to the second LED package is adjusted to be at or near minimum thereby providing illumination depleted in melanopic light.

Some embodiments includes methods of adjusting the spectral output of an LED light engine wherein the means of adjusting the electrical current supplied to the second LED includes a wall dimmer switch. In other embodiments, the means of adjusting the electrical current supplied to the second LED is automated and includes a programmable controller onboard said light engine that adjusts the electrical current. In some embodiments the light engine comprises means for wireless communication.

In some embodiments, the methods of adjusting the spectral output of an LED light engine includes maintaining a near constant color temperature of the illumination output of the light engine during the adjustment of the current flow to the second LED. In still other embodiments, methods of adjusting the spectral output of an LED light engine includes means for generating relatively narrow band illumination in the wavelength band between 410 nm and 430 nm and further comprises the step of generating the narrowband illumination for a time period not exceeding 60 minutes during one or more short portions of the photoperiod.

Embodiments of the invention include a method for providing dynamic and time varying spectral illumination throughout a photoperiod to facilitate circadian rhythm regulation and mitigate social jet lag comprising the steps of: providing a light engine comprising a first LED operable to illuminate high efficacy white light, a second LED operable to produce illumination with a maximum peak intensity between 470 nm and 495 nm and a third LED operable to produce light that has a peak intensity at about 420 nm in the wavelength band between 400 nm and 450 nm, identifying a photoperiod corresponding to at least a portion of a daily human circadian cycle, and adjusting the spectral output of said light engine during said photoperiod to facilitate circadian rhythm regulation wherein the intensity of the illumination output from said second LED is increased and maintained near maximum during a daytime portion of the photoperiod to provide adequate melanopic light and decreased or eliminated during the nighttime portion of the photoperiod and wherein the illumination output of the third LED is temporarily increased for a period of less than one hour at least once during the photoperiod.

Additional embodiments include methods wherein the portion of the daily circadian cycle when the illumination output of the third LED is temporarily increased corresponds to a portion of local dawn or dusk. Embodiments include methods wherein the light engine provided includes a fourth LED package operable to produce illumination enriched with red light and the step of adjusting the spectral output of the light engine during the photoperiod includes increasing the illumination from said fourth LED just prior to increasing the illumination output from the second LED. Other embodiments include methods wherein the light engine provided includes an LED package operable to produce a nighttime spectrum, containing little or no melanopic light and the step of adjusting the spectral output of the light engine includes reducing the output from said first, second and third LEDs and providing illumination from said fourth LED in the evening portion of said photoperiod.

In some embodiments, methods for providing dynamic and time varying spectral illumination throughout a photoperiod to facilitate circadian rhythm regulation and mitigate social jet lag include increasing the illumination output of the third LED temporarily near or during at least one of the portions of the circadian cycle consisting of: the cortisol awakening response, the afternoon lull; the wake maintenance zone. In some embodiments, the increase of the illumination output of said second LED occurs near a wake time of the photoperiod and the decrease of said second LED output occurs within three hours of an estimated sleep time of the photoperiod. In still other embodiments, methods include increasing the illumination output of the second LED gradually such that the intensity of the output increases from minimum to maximum over the time span of at least 45 minutes and wherein the decrease of the illumination output of second LED is gradual and the intensity of the output decreases from maximum to minimum to over the time span of at least 20 minutes.

### BRIEF DESCRIPTION OF THE DRAWINGS

FIGS. 1a-b show, respectively, example spectral power distributions (SPDs) from conventional white light LEDs and the action spectrum of melanopsin and spectral region of blue light hazard overlaid and compared with the spectral power distributions (SPDs) of conventional white light LEDs.

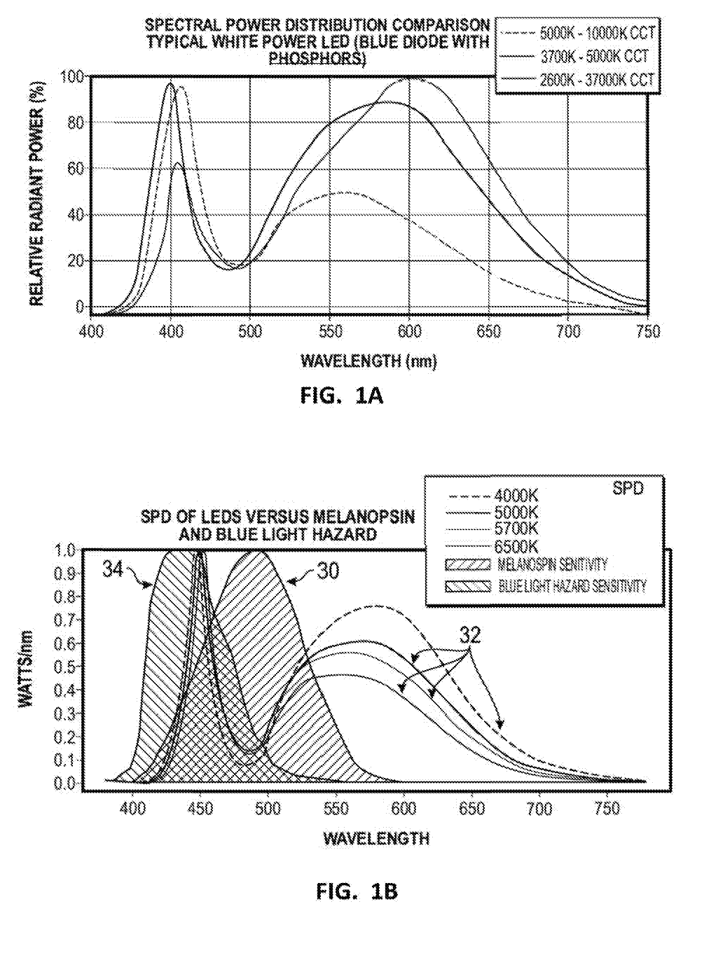

FIG. 2 shows an example schematic illustration of the correlation of circadian rhythms, sleep pressure, sleep and wakefulness.

FIGS. 3a-b shows spectral power densities (SPDs) of SkyBlue® LED packages according to some embodiments.

FIG. 4 show an SPD of a BIOS light engine that includes both a white light LED package and a the SkyBlue LED package according to some embodiments.

FIG. 5 shows examples of SPDs corresponding to the Bio-dimming of an LED light engine according to some embodiments.

FIG. 6 shows a nighttime spectrum according to some embodiments.

FIG. 7 shows a spectral power density plot of the illumination of an LED light package that produces a twilight spectrum according to some embodiments.

FIG. 8 illustrates dynamic lighting and illumination methods that provide spectrally changing lighting throughout a photoperiod to facilitate circadian rhythm regulation according to some embodiments of the invention.

### DETAILED DESCRIPTION

Embodiments of the invention include methods, systems and luminaires that dynamically generate high efficacy white light that comprises enhanced spectral components that vary at different times of the day to facilitate circadian regulation or entrainment. Embodiments of the invention include dynamic illumination methods and systems for providing relatively high melanopic flux during the day and relatively low melanopic flux at night. Other embodiments of the invention include lighting systems which provide for illumination systems that comprise enriched or depleted melanopic light from above such that exposure of melanopic light to photoreceptors in the lower hemisphere of the retina may be amplified or attenuated based on time of day in order to facilitate circadian rhythm regulation.

In some embodiments, a daytime spectrum is generated that has an enhanced circadian spectrum, i.e., melanopic light around 490 nm (or 480 nm-500 nm). In some embodiments illumination includes enhanced spectral components that are relevant to the skin optical window and sub dermal cellular stimulation (e.g., deep-red around 660 nm and/or infrared). Illumination spectrums produced by embodiments of the invention can increase biological stimulus at times where biological sensitivities are greatest. In some embodiments, illumination provided during nighttime will have relatively lower amounts of 480 nm light (i.e., melanopic light), than for example the illumination provided during the daytime. In some embodiments, illumination is produced by, inter alia, pulsing light of particular wavelength regions.

Embodiments of the invention includes systems and luminaires that can alter the illumination spectrum at different times of the day, for examples dynamic systems that can dynamically change the illumination spectrum over the course of a day. In some embodiments relatively higher amounts of deep-red or infrared light (or light in that optical region) are provided during specific times of day to facilitate biological responses including circadian regulation or changes to alertness.

In some embodiments, blue light in the 420 nm region is employed in a lighting system to provide illumination that results in an acute alerting affect. In some embodiments, this illumination is depleted in melanopic light (e.g., light in 490 nm or 460-500 nm) and thereby produces an alerting effect while providing no or reduced impact on the circadian rhythm. The lighting system according to these embodiments produces white light illumination with both high CRI and aesthetic appeal.

Other embodiments of the invention include methods, luminaires and systems for providing biologically relevant light (e.g., melanopic light) from indirect illuminating sources. Embodiments include using white light and/or monochromatic sources, and examples include cove lighting and indirect ceiling and floor lighting. Some embodiments include illumination systems that provide light, that may effect a biological stimulus (e.g., melanopic light), from below such that the light impacts the upper hemisphere of the retina where the opsin photoreceptors are less sensitive thereby reducing the potential biological stimulus. Embodiments include lighting, e.g., indirect light, from above which is depleted of melanopic light but of high CRI thus providing aesthetic white light but without or with reduced biologically stimulating light.

The effect on the circadian cycle as well as on sleep pressure and alerting response of light exposure is one that is highly influenced by daytime biological stimulus including light stimulus. For example, a construction worker who spends most of his days outdoors will experience a smaller impact from light at night compared to someone who spends more of the day in a computer lab with low light levels. This response is dynamic over the course of a day. First morning light helps stimulate cortisol awakening response. Likewise, adaptation for the circadian system is heavily influenced by the light exposure most recently preceding night time or darkness. For example, a high biological light exposure in the late afternoon is also beneficial to circadian regulation and rhythm.

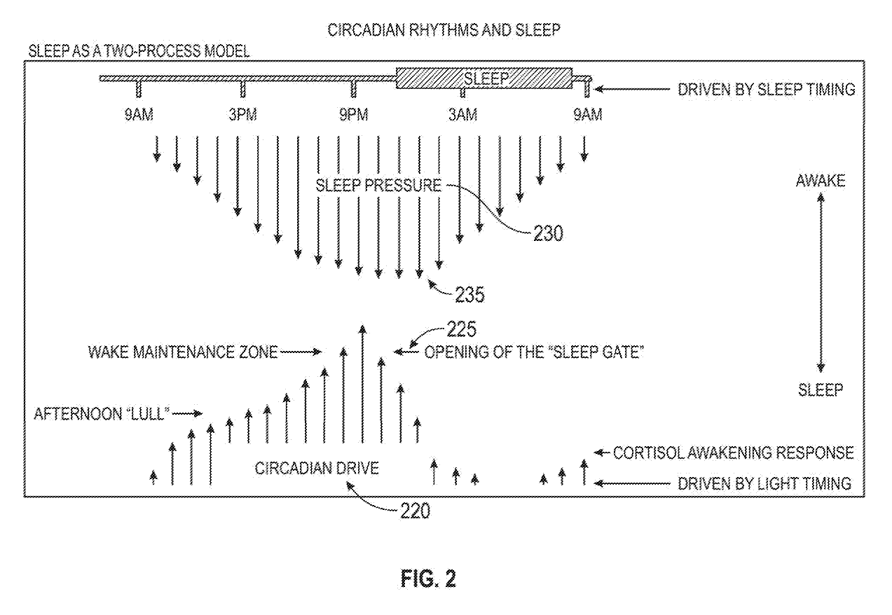

FIG. 2 shows a schematic illustration of a correlation between circadian rhythms and sleep pressure and its relation to the waking-sleep cycle. It is believed that light, both in its intensity and spectral content, plays an important role in circadian rhythm regulation, sleep and wake habits, and alertness levels. A 24 hour period is shown with corresponding circadian drive 220 and sleep pressure 230. The circadian drive 220 represents and shows, inter alia, the state of arousal or “awakeness” throughout the day and the sleep pressure 230 represents the complementary sleep pressure or tendency opposing “awakeness” throughout the same period. The time period start and end times of 9 a.m. is just a common example and for illustrative purposes, and the actual timing and intensities of circadian drives and sleep pressures are variable among individuals and may peak and trough at different times throughout the day. It is believed that the sleep pressure 230 is generally driven, in large part, by the timing of the day, that is in coordination with the circadian rhythm of the individual. The circadian drive 220 is believed to be heavily influenced and driven by the timing and exposure to light. Furthermore, the circadian drives 220 response to light is also dependent on the intensity and spectral content of the light.

As further illustrated in the example shown in FIG. 2, the circadian drive 220 increases during the early part of the day and diminishes as the day progresses. Concomitantly, the sleep pressure 230 also increases and decreases throughout the 24 hour cycles. In a healthy optimum environment, the circadian drive 220 and sleep pressure 230 are relatively synchronized. For instance, the opening of the sleep gate 225 occurs near the same time as the maximum sleep pressure 235 occurs. However, because the circadian drive 220 is influenced by the timing of specific exposure to light, the circadian drive 220 can be shifted due to exposure to light causing the circadian drive 220 and sleep pressure 230 of an individual to “lose synch” causing a disruption in sleep patterns and loss of optimal sleep hygiene. Exposure to light, especially specific kinds and intensities of light, and specific timing of such exposure relative to an individual's circadian rhythm can dramatically influence that individuals circadian rhythm, sleep hygiene and ultimately general health. Embodiments of the invention include systems and methods for providing specific types and intensities of light at specific times of day or light cycle to facilitate and optimize circadian rhythm regulation, improve sleep hygiene and general health. Embodiments include methods of and systems for phase shifting of circadian rhythms

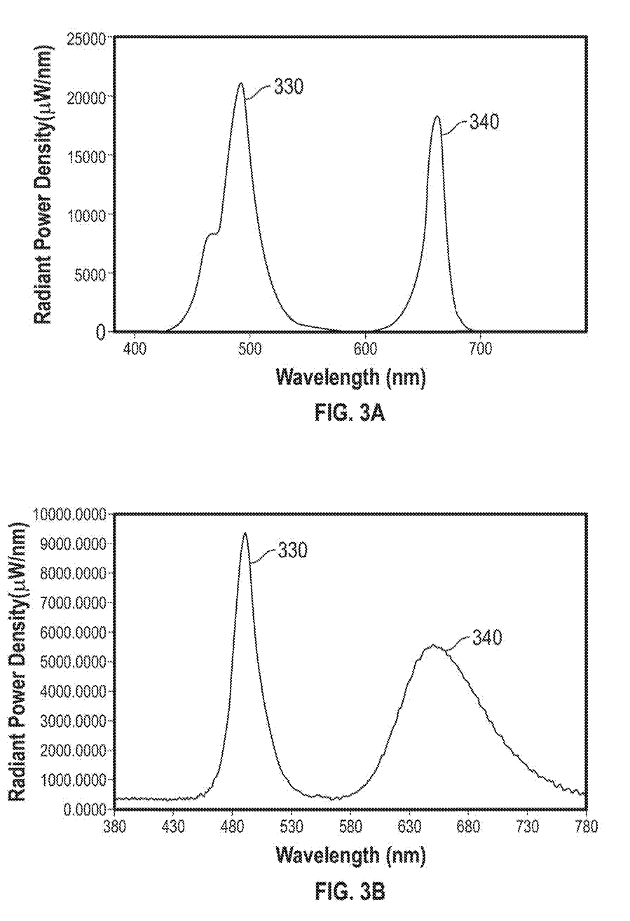

FIGS. 3a-b shows spectral power densities (SPDs) of SkyBlue® LED modules according to some embodiments. The SkyBlue spectrums may be produced by color mixing multiple LED packages or in a preferred embodiment are the result of the illumination from a single LED package, e.g., a pump LED with an associated phosphor that together, when driven to illumination, produce the SkyBlue spectrum(s). The SkyBlue spectrums contain a first peak 330 of power illumination at around 490 nm (e.g., between 470-500 nm) and a second peak 340 centered around 650-670 nm according to these embodiments. The SkyBlue spectrum is preferably used in conjunction with other light packages to provide high efficacy white light with adequate melanopic or biologically effective light. In some embodiments, and as illustrated in FIG. 3b, the peak 330 of power illumination is a relatively narrow band of illumination; in some embodiments, the peak exhibits a FWHM of less than 30 nm. In some embodiments, as shown in FIG. 3a, the second peak 340 is relatively narrow peak with a FWHM of less than or equal to 30 nm; in other embodiments, as shown in FIG. 3b, the second peak 340, centered near 660 nm corresponds to a broader band of illumination. It is to be understood that the relative intensities and spectral widths of the SkyBlue spectrum as shown in FIGS. 3a-b are examples only and a variety of other spectral outputs corresponding to dual peaks, one centered in the melanopic region of the spectrum (e.g., between 480-500 nm) and the other centered near or around 660 nm are contemplated embodiments of the invention.

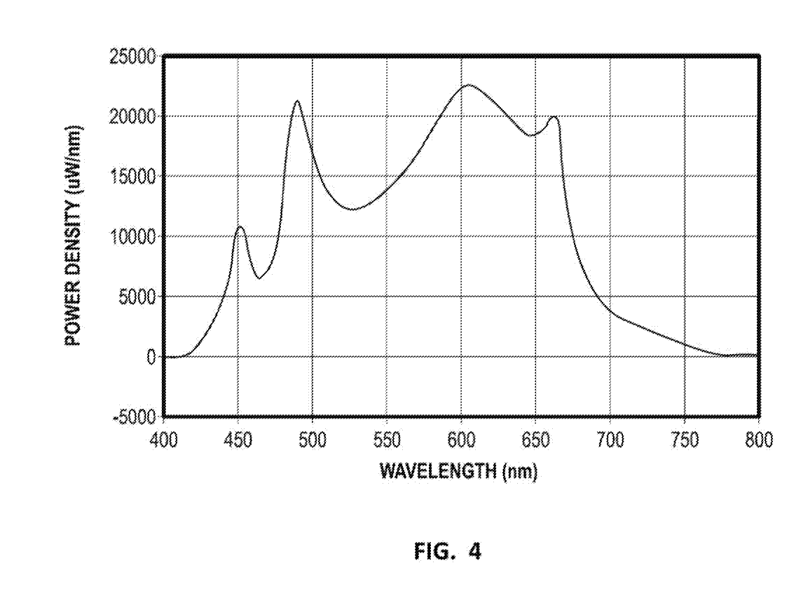

FIG. 4 show an SPD of a BIOS light engine that includes both a white light LED package and a the SkyBlue LED package according to some embodiments. A conventional white LED, e.g., 4000 k is combined with a SkyBlue package and both are electrically driven to illumination to produce the resultant spectrum as shown in FIG. 4. The illumination spectrum is both high efficacy, aesthetically pleasing and is rich in melanopic light. In some embodiments the white light LED and SkyBlue LED packages are electrically driven to yield a single static spectrum as shown. In other embodiments, the intensity of illumination from the SkyBlue package may be adjusted, e.g., via a dimming circuit, to reduce the amount of melanopic light in the resulting light engine spectrum. It is to be understood that the invention is not limited to a specific CCT of white light, and embodiments of the invention include the use of white light LED packages of a variety of color temperatures including but not limited to 2700 K, 3000 K, 3500K, etc. In some embodiments, the SkyBlue spectrum that is mixed with the white light spectrum to produce the spectrum shown in FIG. 4 is the spectrum shown in FIG. 3b.

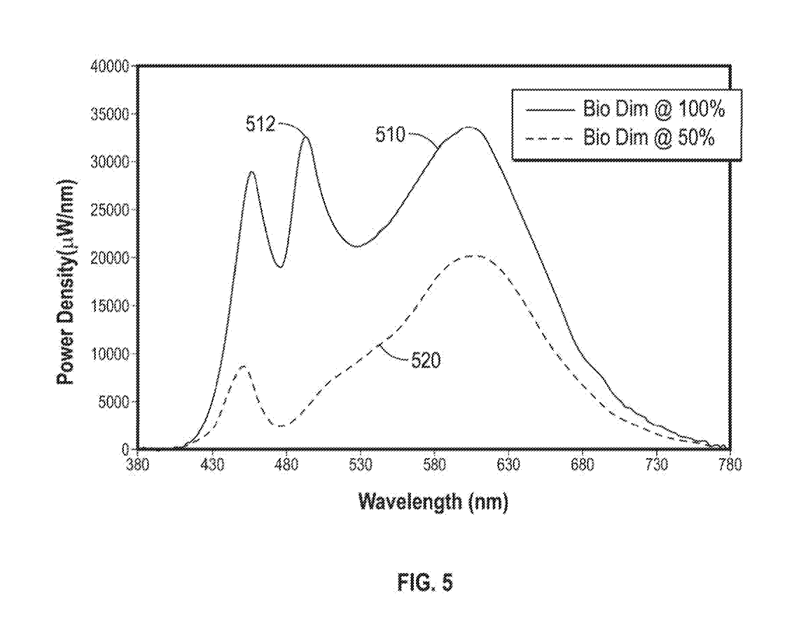

FIG. 5 shows an example of bio-dimming according to some embodiments. FIG. 5 shows SPDs corresponding to an LED light engine containing a SkyBlue LED package and a white light LED package wherein the illumination from the SkyBlue package may be selectively varied using dimming circuitry (not shown). The dimming circuitry provides for variation (e.g., reduction) in the electrical current to the SkyBlue package thereby reducing illumination from the SkyBlue package according to some embodiments. Spectrum 510 of the light engine corresponds to the illumination output of the light engine when the SkyBlue LED package is fully energized and spectrum 520 corresponds to the illumination output of the light engine when the current to the SkyBlue LED package has been reduced (e.g., by 50%). According to these embodiments, the light engine comprises both a high efficacy white light package (e.g., 3500 K or 4000K—although the invention is not limited to any specific white CCT) and a SkyBlue package (for producing a SkyBlue spectrum, for example as shown in FIGS. 3a-b). As shown in 510, when the SkyBlue package is fully energized (100%), the output spectrum contains an emission peak 512 centered near 490 nm (i.e., rich in melanopic light) whereas when the light engine is bio-dimmed, that is the current to the SkyBlue LED package is reduced, e.g., by 50%, the emission in the melanopic region is greatly reduced and the output spectrum does not exhibit a peak in the melanopic region (e.g., 470-500 nm). Embodiments of the invention include dimmable light engines as described above that illuminate with varying amounts of melanopic light according to the dimming level. It is to be understood that the spectra shown in FIG. 5 are mere example embodiments and are not meant to be limiting. As will be evident to those skilled in the art, a variety of dimming levels and protocols allow for the fine tuning of the amount of melanopic light produced by the light engine (e.g., 90% of maximum, 10% of maxim, 0% maximum, etc) such that the amount of melanopic light can be varied in intensity throughout the day or other photoperiod (e.g., circadian day). Embodiments of the invention include light engines with onboard dimming circuitry such that power delivered to the SkyBlue LED package may be selectively reduced and the SkyBlue package effectively dimmed thereby reducing the amount of melanopic light produced by the light engine.

In some embodiments a conventional 0-10 V dimmer switch is used to adjust the electrical current to the SkyBlue package thereby controlling the amount of the Skyblue spectral component in the overall illumination of the light engine. By using the conventional dimming circuitry, the amount of SkyBlue spectrum is adjusted thereby increasing or decreasing the melanopic component of the resulting illumination. 510 is an SPD of the light engine where the SkyBlue component is not dimmed at all; SPD 510 is rich in melanopic light and appropriate for, inter alia, daytime lighting. 520 is an SPD showing an example of Bio-dimming wherein the intensity of the illumination from SkyBlue package has been reduced by 50% (e.g., current from the dimmer is set at 5 V) and the SkyBlue spectral component has been reduced in intensity. As shown in SPD 520, the amount of melanopic light has been greatly reduced. The Skyblue component spectrum can be reduced to zero with and appropriate dimmer setting thereby elimination all the melanopic light. Such a dimming level may be appropriate prior to bedtime.

Other embodiments include a bio-dimmable light engine that is linked to a clock and which automatically dims or adjusts the amount of SkyBlue component and thus melanopic light throughout the day to coordinate and facilitate circadian rhythm regulation. In some embodiments, biological dimming is accomplished using a 0-10 V wall dimmer switch. When the switch is set on maximum, i.e., 10 V, the SkyBlue component is at full intensity and decreasing the dimmer setting towards 0 V reduces the radiance from the SkyBlue component (i.e., decreases the melanopic light). In some embodiments, the color temperature is altered during dimming.

In other embodiments, the color temperature is maintained relatively constant while dimming. Embodiment variations include a light engine containing an additional LED package that emits in the 410-450 nm spectral region and which can be selectively driven to illumination via the dimmer switch or circuitry. Light in this spectral region has an acute alerting effect while not significantly impacting circadian drive and so can be used to “wake up” or increase arousal level while not disrupting circadian rhythms.

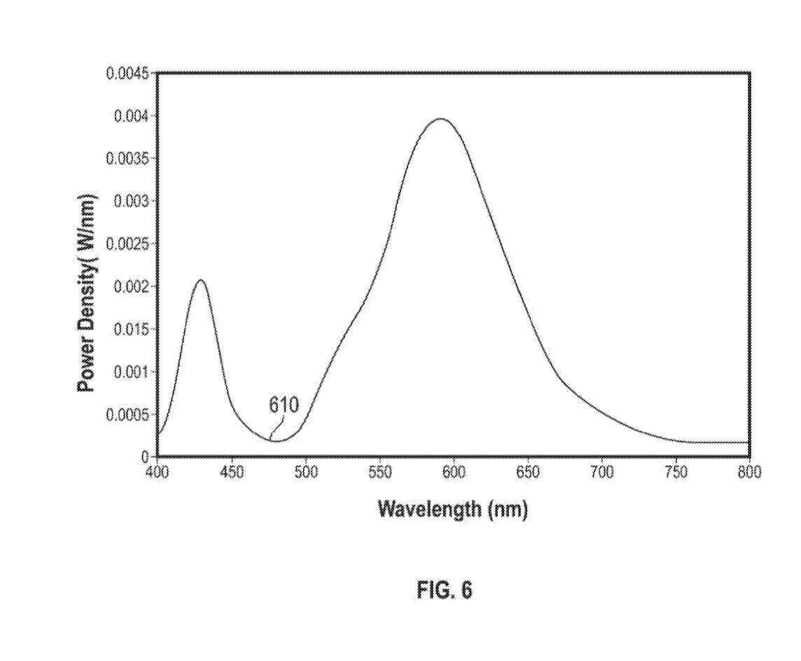

FIG. 6 shows a nighttime spectrum according to some embodiments. The nighttime spectrum has very little melanopic light as shown by the trough 610 in spectral intensity between 450 and 500 nm. In some embodiments, the nighttime spectrum results from a complete dimming of the SkyBlue spectral component in a light engine comprising both a white light LED and a SkyBlue LED. In other embodiments, the nighttime spectrum is contained within a single LED package and may be used as a single channel nighttime light.

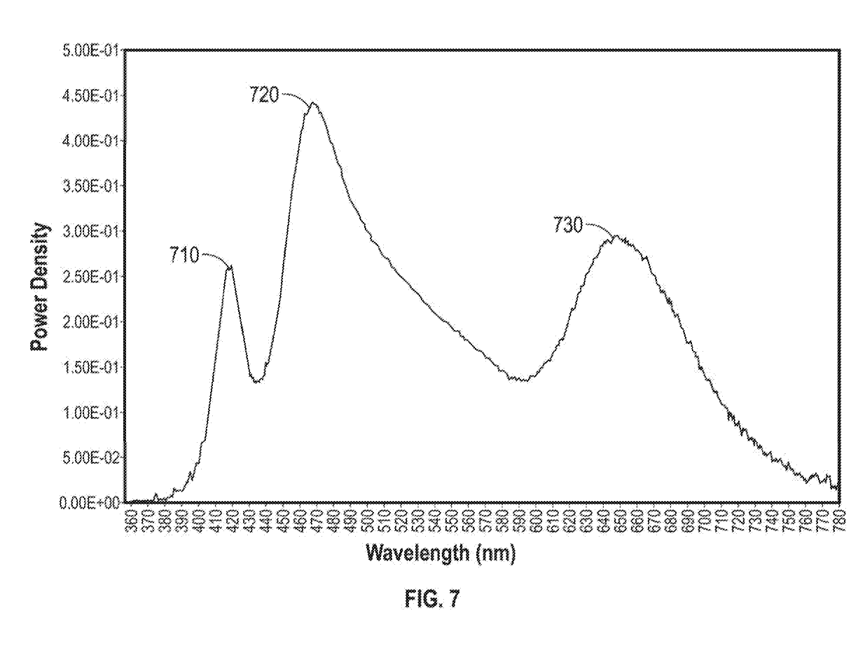

FIG. 7 shows a spectral power density plot of the illumination of an LED light package that produces a twilight spectrum according to some embodiments. The twilight spectrum includes a peak 710 at or around 420 nm. The twilight spectrum also includes a peak 720 in the 465 nm region, and another peak 730 centered near the 660 nm region (640-680 nm). Peak 720 corresponds to light that maximally suppresses melatonin. In some embodiments, the twilight spectrum is generated by a light engine comprising multiple LED dies or chip of different colors. In these embodiments, the LEDs are essentially color mixed in order to produce the twilight spectrum. In other embodiments, the twilight spectrum is produced by a single LED package. The single LED package is fabricated using a choice selection of blue pump LEDs in conjunction with specific phosphor combinations. The twilight spectrum has a blue hue that may have a significant biological impact in terms of helping the body circadian system delineate between daytime and nighttime.

Embodiments of the invention include LED lighting systems that provide automated spectral control of illumination throughout day (or other photoperiod) to facilitate circadian rhythm regulation, optimize sleep hygiene, and help mitigate social jet jag. Embodiments of the invention include lighting systems that produce dynamic spectrums which have a heightened amount of 420 nm and a reduced or minimal amount of 49 Onm during the beginning and the end of the daytime photoperiod. Embodiments include dynamic lighting that illuminates with red light prior to significant illumination with the melanopic light (e.g., 490 nm) in order to potentially amplify the human neurological response of melanopsin. In some of these embodiments, light with an enriched red component is provided just prior to light with the enriched melanopic light. In some other embodiments, red enriched light is provided after the illumination with a 420 nm rich twilight spectrum and prior to illumination with the 490 nm rich daytime spectrum. It is believed that such exposure to light enriched with red light prior to exposure to melanopic rich light will enhance human circadian signaling factors. In some embodiments the enriched red light is produced using a monochromatic LED. In other embodiments, the red light is created from a phosphor or quantum dot down conversion. Embodiments of the invention include dynamic lighting systems which begins the day with a heightened amount of 420 nm, followed by a heightened amount of red stimulation, followed by a heightened amount of 490 nm, followed by a heightened amount of red light followed by a heightened amount of 420 nm light, followed by a biological low stimulating nighttime light. Other embodiments of the invention do not include the red portion of this dynamic spectrum process.

Embodiments of the invention include a multi-channel light engine comprising select LED packages that is selectively electrically driven and operable to illuminate with varying spectral outputs throughout the course of the day or other photoperiod. In some embodiments, the LED light engine comprises a white light LED package (e.g., 3500 K, 4000K, or 5000K), a SkyBlue LED package (an LED package that illuminates the SkyBlue spectrum as shown in FIG. 2) and a Twilight LED package (an LED package that illuminates the SkyBlue spectrum as shown in FIG. 6). Embodiments include a control system for adjusting the output spectrum of the light engine. The control system may be manual, for example a wall dimmer switch or wireless smart phone control. In other embodiments, the control system is automated and controlled by an onboard or remote processor and may be pre-programmed to run without user input, for example in coordination with a local clock or other preprogrammed instructions. In wireless embodiments, the light engine includes or is couple to a received antenna for receiving external commands or program instructions. In some of these embodiments, the amount of illumination coming from each of the LED packages may independently varied with time to alter the overall output spectrum of the LED light engine throughout the day or other photoperiod. For example, during the daytime, the SkyBlue spectrum may be ramped up to supply enhanced melanopic light whereas in the evening, the intensity of the SkyBlue spectrum may be reduced or eliminated. In another example, the Twilight spectrum may be ramped up for a short time at the beginning and/or end of the day in order to simulate dawn or dusk. Some embodiments include only a white light LED package and a SkyBlue package. Other embodiments include an additional red light LED package. Some embodiments include a nighttime LED package that illuminates with a spectrum as shown in FIG. 5.

The lighting system according to some embodiments comprises one or more luminaires or light sources that illuminate the environment of one or more individuals throughout the photoperiod, and which are dynamically adjusted throughout the photoperiod to provide varying and appropriate spectral outputs. This dynamic spectrally controlled illumination throughout the photoperiod may be used to facilitate regulation of circadian rhythms, maintain alertness, enhance sleep hygiene and generally improve personal health. It may also be used to align the circadian rhythms of a population of individuals who are exposed to the same patterns of illumination. In some embodiments, the luminaires or lighting fixtures of the system may be distributed across different rooms or buildings and the lights may be synchronized to a common clock in order to provide the appropriate spectral/temporal output.

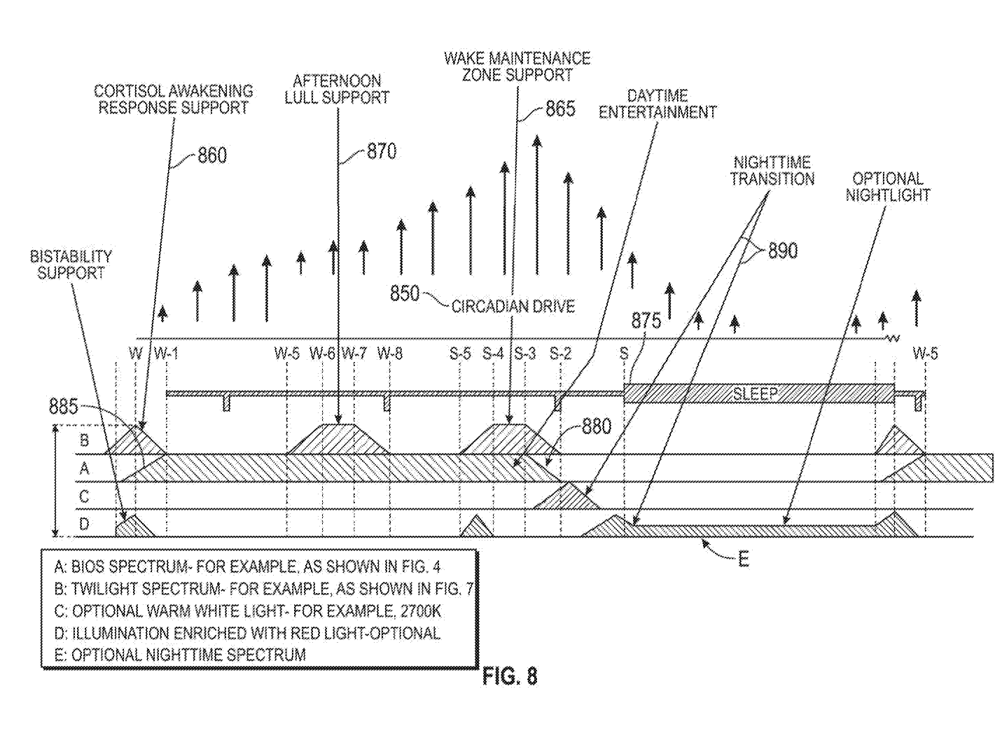

FIG. 8 illustrates an example of how embodiments of the invention provide spectrally dynamic lighting throughout a photoperiod. In the example illustrated, the photoperiod corresponds to a day (e.g., a one circadian cycle). A typical circadian period is typically around 24 hours, but can vary slightly amongst individuals. The time evolution of the photoperiod day is represented along the horizontal axis and the time units are delimited as hours before or after an individual wakes from sleep. For example, W represents the time of waking, and W+1 and W+8 represent the times 1 hour and 8 hours after waking respectively. Similarly W−5 represents the time 5 hours prior to waking. S represents the time of sleep onset, and the times S−2 and S−5 represent the times 2 hours and 5 hours before the onset of sleeping respectively. Also shown in FIG. 8, along the vertical axis, is the circadian drive 850 (as in FIG. 2). The level of circadian drive varies throughout the day photoperiod and is schematically represented by the length of the vertical arrows. Specific types and quantity of light exposure during various points of the photoperiod can dramatically affect the circadian cycle. The proper type of light at the proper time may facilitate a smooth circadian rhythm, healthy sleep hygiene and other beneficial biological effects. Conversely, the wrong type of artificial light at the wrong time can disrupt the circadian cycle and interfere with sleep patterns and general health.

Examples of dynamic spectral output of light engines and luminaires according embodiments of the invention are shown in FIG. 8. In this example, the Twilight spectrum (B) is ramped up during the period near waking, e.g., W+/−1 hour to coincide with and support the Cortisol Awakening Response 860. Likewise, the twilight spectrum (B) is again ramped up at the end of the day, for example at 3-5 hours prior to the expected onset of sleep, in order to coincide with and support the Wake Maintenance Zone 865. The increased exposure to the 420 nm light may provide an acute alerting effect. The Twilight spectrum (B) is maintained only for a brief time, e.g., 30-45 minutes to correspond to the twilight period of the day according to these embodiments. The twilight spectrum may also be ramped up briefly during midday to support and provide alerting light during the “afternoon lull” 870 that typically occurs during the day and is associated with reduced wakefulness.

According to some embodiments and as shown in FIG. 8, during the main part of the day, that is right after waking up and up to within 2-3 hours of initiating sleep, illumination with the BIOS spectrum (A) is provided (e.g., the spectrum as shown in FIG. 3). The BIOS spectrum is produced according to some embodiments from the combination of a white light LED (e.g., 2700 L, 3500 K, 4000 K, etc) and a SkyBlue LED package that is rich in melanopic as discussed elsewhere herein. As the photoperiod approaches the sleep portion of cycle 875, the SkyBlue spectral component is dimmed such that the melanopic light is reduced or eliminated in advance of and to prepare for sleep. FIG. 8 shows the ramping down 880 of the SkyBlue spectral component between times S−3 and S−2 such that there is no melanopic light 2 hours prior to sleep time. Similarly and according to some embodiments, the SkyBlue spectrum is ramped up 885 beginning at the time of waking W to maximum intensity at W+1 and maintained there until the ramp down 880 at S−3. The illumination throughout the day with the SkyBlue enhanced spectrum facilitates circadian entrainment.

After the Skyblue spectrum has been ramped down (or coinciding with its ramp down) a ramping up of one or night nighttime spectrum may be employed to maintain light level or provide aesthetic warm light for evening time. This nighttime transition 890 can be achieved using an optional warm white light package (C), e.g., 2700 K white light. Alternatively or additionally, illumination from a nighttime LED package may be used during the pre-sleep period or as a nightlight during the sleep period. An example embodiment of a nighttime spectrum E is shown in FIG. 6. An optional enriched red spectrum D may also be used for bi-stability support at various points in the photoperiod as discussed above. In the example shown, red enriched light D is provided just prior to the ramping up of the SkyBlue component or the Twilight component or both.

Although multiple spectral outputs corresponding to multiple LED packages are shown in the example of FIG. 8, embodiments of the inventions are not limited to the specific combinations or spectrums shown. Embodiments of the invention may include a subset of these outputs or additional outputs. Also, although the intensity of the various spectra are not illustrated in FIG. 8, it is to be understood that the intensity of the individual spectra is a parameter of the system and the intensities of one of more of the illumination outputs such as the SkyBlue or Twilight spectrums may be adjusted to achieve the desired total spectral illumination (see, for instance, Bio-dimming as discussed above).

While the invention has been described with reference to exemplary embodiments, it will be understood by those skilled in the art that various changes may be made and equivalents may be substituted for elements thereof without departing from the scope of the invention. It should be understood that the diagrams herein illustrates some of the system components and connections between them and does not reflect specific structural relationships between components, and is not intended to illustrate every element of the overall system, but to provide illustration of the embodiment of the invention to those skilled in the art. Moreover, the illustration of a specific number of elements, such as LED drivers power supplies or LED fixtures is in no way limiting and the inventive concepts shown may be applied to a single LED driver or as many as desired as will be evident to one skilled in the art.

In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include many variants and embodiments. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. An LED light engine for producing illumination with adequate amounts of melanopic light and for facilitating circadian rhythm regulation comprising:
a first LED module operable to produce white light illumination; and
a second LED module operable to produce illumination with a first peak intensity between 470 nm and 500 nm and a second peak intensity between 640 nm and 680 nm wherein the second peak intensity is less than said first peak intensity.

2. The LED light engine of claim 1 further comprising a third LED module operable to produce illumination with a first peak intensity between 455 nm and 475 nm and a second peak intensity between 410 nm and 430 nm.

3. The LED light engine of claim 2 further comprising electrical circuit means for connecting said first and second and third LED modules to a source of electrical power whereby the magnitude of electrical power supplied to said second and third LED modules may be varied thereby varying the intensity of the illumination output of said second and third LED modules.

4. The LED light engine of claim 2 wherein the correlated color temperature of the output from the light engine when both the second LED module and third LED module are energized to illumination exceeds 7500 K.

5. The LED light engine of claim 1 wherein said first LED package produces white light with a correlated color temperature between 2500 K and 3500 K.

6. The LED light engine of claim 1 wherein the full width of the peak at half its maximum intensity of said first peak intensity of said second LED package is less than 30 nm.

7. The LED light engine of claim 1 further comprising a nighttime LED module operable to emit light wherein the total radiant power emitted in a first wavelength band from 400 nm to 450 nm is greater than 8% of the total radiant power emitted and wherein the total radiant power emitted in a second wavelength band from 450 nm to 500 nm is less than 3% of the total radiant power emitted.

8. A method of adjusting the spectral output of an LED light engine to facilitate circadian rhythm regulation comprising the steps of:
providing a light engine comprising a first LED module and a second LED module wherein the first LED module produces white light and the second LED module produces light that has a maximum peak emission intensity between 470 nm and 490 nm and wherein the light engine contains means for adjusting electrical current supplied to said second LED module; and
adjusting the current flow to said second LED package such that the intensity of light emitted from the light engine between 470 nm to 490 nm is increased during a first portion of a photoperiod and decreased during a second portion of the photoperiod.

9. The method of claim 9 wherein said first portion of the photoperiod corresponds to circadian daytime and the current flow to the second LED package is adjusted to be at or near maximum thereby providing illumination rich in melanopic light and wherein said second portion of the photoperiod corresponds to circadian nighttime and the current flow to the second LED package is adjusted to be at or near minimum thereby providing illumination depleted in melanopic light.

10. The method of claim 8 wherein the means of adjusting the electrical current supplied to the second LED includes a wall dimmer switch.

11. The method of claim 8 wherein the means of adjusting the electrical current supplied to the second LED is automated and includes a programmable controller onboard said light engine that adjusts the electrical current and wherein the light engine comprises means for wireless communication.

12. The method of claim 8 further comprising the step of maintaining a near constant color temperature of the illumination output of the light engine during the adjustment of the current flow to the second LED.

13. The method of claim 8 wherein the light engine further includes means for generating relatively narrow band illumination in the wavelength band between 410 nm and 430 nm and further comprising the step of generating said narrowband illumination for a time period not exceeding 60 minutes during one or more portions of the photoperiod.

14. A method for providing dynamic and time varying spectral illumination throughout a photoperiod to facilitate circadian rhythm regulation and mitigate social jet lag comprising the steps of:
providing a light engine comprising a first LED operable to illuminate high efficacy white light, a second LED operable to produce illumination with a maximum peak intensity between 475 nm and 495 nm and a third LED operable to produce light that has a peak intensity at about 420 nm in the wavelength band between 400 nm and 450 nm;
identifying a photoperiod corresponding to at least a portion of a daily human circadian cycle; and
adjusting the spectral output of said light engine during said photoperiod to facilitate circadian rhythm regulation wherein the intensity of the illumination output from said second LED is increased and maintained near maximum during a daytime portion of the photoperiod to provide adequate melanopic light and decreased or eliminated during the nighttime portion of the photoperiod and wherein the illumination output of the third LED is varied from a first low level to a second higher level and then from the second higher level back to a lower level all over period not exceeding one hour at least once during the photoperiod.

15. The method of claim 14 wherein the portion of the daily circadian cycle when the illumination output of the third LED is temporarily increased corresponds to a portion of local dawn or dusk.

16. The method of claim 14 wherein the light engine provided includes a fourth LED package operable to produce illumination enriched with red light and the step of adjusting the spectral output of the light engine during the photoperiod includes increasing the illumination from said fourth LED just prior to increasing the illumination output from the second LED.

17. The method of claim 14 wherein the light engine provided includes an LED package operable to produce a nighttime spectrum, containing little or no melanopic light and the step of adjusting the spectral output of the light engine includes reducing the output from said first, second and third LEDs and providing illumination from said fourth LED in the evening portion of said photoperiod.

18. The method of claim 14 wherein the illumination output of the third LED is temporarily increased near or during at least one of the portions of the circadian cycle consisting of: the cortisol awakening response, the afternoon lull; the wake maintenance zone.

19. The method of claim 14 wherein the increase of the illumination output of said second LED occurs near a wake time of the photoperiod and the decrease of said second LED output occurs within three hours of an estimated sleep time of the photoperiod.

20. The method of claim 19 wherein the increase of the illumination output of second LED is gradual and the intensity of the output increases from minimum to maximum over a time span of at least 45 minutes and wherein the decrease of the illumination output of second LED is gradual and the intensity of the output decreases from maximum to minimum to over a time span of at least 20 minutes.
